(19) United States
(12) Patent Application Publication
(10) Pub. No.: US 2001/0018090 A1
(43) Pub. Date: Aug. 30, 2001

(54) Calorie reducing agent

(76) Inventors: Shinzo Noda, Kanagawa (JP); Shigenobu Mizusaki, Kanagawa (JP)

Correspondence Address: FLYNN, THIEL, BOUTELL & TANIS, P.C., 2026 Rambling Road, Kalamazoo, MI, 49008-1699, US

(21) Appl. No.: 09/764,497

(22) Filed: Jan. 17, 2001

(30) Foreign Application Priority Data
Jan. 28, 1999 (JP) 11-20081

(51) Int. Cl. A23F 3/00

(52) U.S. Cl. 426/597; 426/2; 424/439; 514/783; 514/909

(57) ABSTRACT

To provide a calorie reducing agent, a calorie reducing food/drink and a calorie reducing feed for animals manufactured from food which can be usually ingested. 1-Deoxynojirimycin or an analogue thereof is used as an effective ingredient for the calorie reducing agent.

### FIELD OF THE INVENTION

The present invention relates to a novel use of a substances derived from plants of family Moraceae. To be more specific, the present invention relates to a calorie reducing agent where 1-deoxynojirimycin, an analogue thereof, mulberry tea manufactured from plants of family Moraceae containing 1-deoxynojirimycin an analogue thereof, a powdered product or an extract thereof is an effective ingredient and also to a calorie reducing food/drink and a feed for animals where carbohydrate represented by starch and sugar is added thereto.

### BACKGROUND ART

In recent years, our daily food life has been significantly improved and onset of the so-called a life-related disorder (an adult disease) due to an excessive ingestion of calorie is much talked about now. Particularly, diabetes mellitus which causes serious complications such as retinopathy, nephropathy and neuropathy is becoming a social problem spreading not only among adults but also among children. It is said that, in the therapy of diabetes, a combination of dietary therapy and physical exercise therapy together with a therapy by administration of pharmaceuticals is important.

The thing which is utilized for preparing the menu in conducting the dietary therapy is a food exchange table. In the food exchange table, foods are classified into six groups—cereals, vegetables, fruits, milks, meat/fish/egg and fat/oil—and units for each food is determined where 1 unit is 80 kcal. In preparing the menu, a combination of food groups is determined on the basis of the units depending upon the daily ingestion calorie of a patient.

In the therapy of diabetes, it is directed that ingestion of calorie per day is to be kept at 1300-1600 kcal. For example, when a menu for ingestion of 1600 kcal per day is to be drafted, it is a simple idea that each of three meals is made 6.7 units since 1600 kcal corresponds to 20 units. When one meal (6.7 units) is divided into 3.5 units of a staple dish and 3.2 units of a subsidiary dish, 193 g (a little under two bowls of medium size) of rice can be taken as a staple food. With regard to 3.2 units of the subsidiary dish, side dish is to be suppressed to 1.8 units because 200 ml (one bottle) of milk corresponds to 1.4 units if it is taken. Alternatively, if one wants to take one mandarin orange (80 g) and 13 ml of wine, they correspond to 0.26 unit and 0.11 unit, respectively, and their total is 0.37 unit whereby 2.72 units of a side dish can be taken.

It is usually said that a well-balanced means are available when a menu is prepared from 30 kinds of food per day and also the ratio (by weight) of saccharide:protein:lipid is made 55-60:15-20:20-25. However, it is often difficult to assemble them from the above-mentioned small units and it sometimes happens that the menu lays an emphasis on calorie control rather than on well-balanced food whereby many patients are compelled to spend their dietary life with patience.

Persons suffering from diabetes have a strong hunger feel to sweet taste and the menu where seasoning is made as plain as possible and sweet taste is suppressed as much as possible often results in stress of the persons taking such a food. According to the food exchange table, use of 6 g (0.3 unit) of sugar as a seasoning per day is taught to be appropriate but it frequently happens that the patient does not satisfy such a dose but is apt to take much more sugar.

For the prevention of the life-related disorder, “Instruction for Dietary Life” (by the Ministry of Health and Welfare) says that it is desirable to suppress the ingesting amount of sugar to 40-50 g per day. However, the commercially available canned drink (a 350-g can) which we usually take contains about 21-36 g (27 g in average) of sugar and, therefore, taking only two canned drinks per day exceeds the recommended ingesting amount.

The calorie of 27 g of sugar contained in one canned drink corresponds to 108 kcal. For consuming that energy, it is necessary to conduct an exercise of about 14-17 minutes by rope skipping or about 18-22 minutes by jogging. If sugar is taken from other foods, much more time for physical exercise is required.

In that case, a substitute sweetener may be used for reduction of calorie. With regard to such a substitute sweetener, there have been used sorbitol, fructose, maltitol, saccharine, stevia, aspartame and the like. However, although sorbitol is used as a substitute sweetener because of the reason that its slow absorption does not cause a sudden rise of blood glucose level, it is utilized via a usual metabolic route and, therefore, its calorie per gram is 4 kcal which is as same as that of sugar. There is another disadvantage that its sweetness is as low as only 60-70% of sugar. Fructose has a difficulty in actual use that it is unstable at high temperature and its sweetness decreases at high temperature and increases at low temperature. In the case of maltitol, adverse action such as diarrhrea may be resulted when ingested in a large amount. In saccharine, its sweetness has a bitter taste. In stevia, expression of sweetness is somewhat slow leaving an unpleasant aftertaste. Aspartame has a disadvantage that it has no mellow taste of sugar and the aftertaste is bad as well. As compared with the substitute sweeteners having organoleptic disadvantages as such, sugar has the characteristics that its sweet taste is unchanged even when the cooking temperature is changed being able to maintain stable and natural sweetness. Accordingly, it is said that sugar is a fundamental sweetener used for food.

The important thing in the dietary therapy is a between-meal snack. The between-meal snack is often confectionery. The calorie source of the confectionery is mostly occupied by sugar as noted in wagashi (Japanese cake) and cake/candy/cookie, etc. Since confectionery has a good taste, it is often taken too much resulting in an over-calorie. However, reduction of sugar causes elimination of the natural sweet taste whereby the good taste as a food is lost.

Under such circumstances, there has been a demand for the production of food where the calorie is suppressed and the natural sweetness is still maintained.

In view of the above, there has been a demand for a calorie reducing agent by which, even when the food having a good taste prepared by the use of sugar or the like having natural sweetness is excessively ingested, such nutrients do not cause an over-calorie.

### SUMMARY OF THE INVENTION

In order to solve the above-mentioned problems, the present inventors have repeatedly carried out intensive studies and, as a result, they have found that, when mulberry is ingested, a calorie reducing effect is achieved due to suppression of absorption of carbohydrates into body whereupon the present invention has been accomplished.

Thus, the present invention relates to a calorie reducing agent which is characterized in containing a substance derived from plants of family Moraceae represented by mulberry tea, particularly, 1-deoxynojirimycin as an effective ingredient and also to a calorie reducing food/drink and animal feed where carbohydrate or a carbohydrate-containing food is added thereto.

The invention relates to a method of reducing a calorie of food or beverage, which comprises administering an effective amount of 1-deoxynojirimycin or an analogue thereof as the effective ingredient to a person or animal.

The effective amount of 1-deoxynojirimycin or an analogue thereof may be incorporated into food or beverage.

The administration may be effected together with food or beverage. It maybe effected alternatively before or after eating, within about 30 minutes before or after. The method of the invention may be effective during digestion.

The effective substance, that is, the calorie reducing agent, is preferably selected from 1-deoxynojirimycin, an analogue thereof, a mulberry tea manufactured from a plant of family Morac containing 1-deoxynojirimycin or an analogue thereof, a powdered plant of family Moraceae containing 1-deoxynojirimycin or an analogue thereof and an extract of a plant of family Moraceae containing 1-deoxynojirimycin or an analogue thereof.

The above shown methods may be effected by incorporating also a carbohydrate such as sugar and starch.

The invention provides a calorie reducing composition comprising a carbohydrate and at least one substance selected from the group consisting of the above shown calorie reducing agents.

The method of the invention can be applied to human being or animal such as domestic animal.

In the invention, 1-deoxynojirimycin or an analogue thereof may be applied in the form of a mulberry tea manufactured from a plant of family Moraceae containing 1-deoxynojirimycin, a powdered plant of family Moraceae containing 1-deoxynojirimycin or an extract of a plant of family Moraceae containing 1-deoxynojirimycin.

In the invention, a 1-deoxynojirimycin analogue may be used. Examples are:

2,5-dihydroxymethyl-3,4-dihydroxypyrrolidine

1-deoxymannojirimycin

β-homonojirimycin

α-homomannojirimycin

β-homomannojirimycin

α-3,4-di-epi-homonojirimycin

adenophorine

1-deoxyadenophorine

5-deoxyadenophorine

α-1-C-ethylfagomine

fagomine

4-O-β-D-glucopyranosylfagomine

3- O-β-D -glucopyranosylfagomine

3-epifagomine

2,5-dideoxy-2,5-imino-D-mannitol

N-methyl-1-deoxynojirimycin

calystegin C₁

calystegin B₂

1,4-dideoxy-1,4-imino-D-arabinitol

1,4-dideoxy-1,4-imino-D-ribitol

5-hydroxymethyl-3,4-dihydroxypyrrolidine

β-1-C-butyl-deoxygalactonojirimycin

2,5-dideoxy-2,5-imino-DL-glycero-D-manno-heptitol

2,5-imino-2,5,6-trideoxy-D-manno-heptitol

2,5-imino-2,5,6-trideoxy-D-gluo-heptitol

1-O-β-D-glucopyranosyl-1-deoxynojirimycin

2-O-β-D-glucopyranosyl-1-deoxynojirimycin

3-O-β-D-glucopyranosyl-1-deoxynojirimycin

4-O-β-D-glucopyranosyl-1-deoxynojirimycin

1-O-α-D-glucopyranosyl-1-deoxynojirimycin

2-O-α-D-glucopyranosyl-1-deoxynojirimycin

3-O-α-D-glucopyranosyl-1-deoxynojirimycin

1-O-α-D-galactopyranosyl-1-deoxynojirimycin

4-O-α-D-galactopyranosyl-1-deoxynojirimycin

β-D-glucopyranosyl-adenophorine

β-D-glucopyranosyl-5-deoxyadenophorine

7-O-β-D-glucopyranosyl-α-homonojirimycin

5-O-α-D-galactopyranosyl-α-homonojirimycin

5-hydroxymethyl-2-O- (β-D-glucopyranosyl) -3,4-dihydroxypyrrolidine

### DESCRIPTION OF THE PREFERRED EMBODIMENTS

The present invention will now be illustrated in detail as hereunder.

In plants, 1-deoxynojirimycin which is an effective ingredient in the present invention is contained in mulberry and its presence has not been confirmed in the plants other than mulberry. In the present invention, it is not always necessary to use by extracting 1-deoxynojirimycin in a pure state but it goes without saying that the form derived from the plant of family Moraceae containing 1-deoxynojimycin, i.e. the plant as it is or a form of powdered or extracted product thereof, may be used as well.

With regard to the material used in the present invention, there is no particular limitation so far as it is the plant of family Moraceae and, for example, Morus bombycis Koidzumi, Morus alba L., Morus australis Poir, Morus latifolia Poir, Morus mongolica Schneid., Morus nigra L., Morus rubra L. and Morus boninensis Koidzumi may be used. Crossbreeds and variants thereof may be used as well. The parts to be used are not limited at all but any of leaves, branches, young branches, barks, stems, roots, peelings of roots, flowers, mulberry fruits, etc. may be used.

For example, in the manufacture of a mulberry leaf extract containing 1-deoxynojirimycin, water or alcohol such as an aqueous ethyl alcohol is added to dried mulberry leaves to extract. The dried mulberry leaves used as the material are not limited at all but, in addition to the dried material itself, mulberry leaves roasted with a roaster may be used as well. It is also possible to use powder which is prepared by powdering the dried material using a pulverizer. The extract is treated with a centrifuge to remove insoluble matters and then concentrated in vacuo to give a mulberry leaf extract. The resulting extract contains 0.4-1% by weight of 1-deoxynojirimycin.

After that, the extract is repeatedly subjected to a treatment with an ion-exchange resin to give crystalline 1-deoxynojirimycin.

Furthermore, 1-deoxynojirimycin which is used in the present invention is not limited to that which is extracted and isolated from the plants of family Moraceae but a substance prepared by a chemical synthetic method or a fermentation method or by using microorganisms may be used as well.

1-Deoxynojirimycin is a sugar-like alkaloidal substance contained in the plants of Moraceae and is a polyhydroxypyrrolidine derivative having a fundamental structure where oxygen of a pyranose ring of glucose is substituted with nitrogen. It is contained in an amount of 0.1-0.2% by weight in dried mulberry leaves.

In the present invention or in the case where a substance derived from the plants of Moraceae containing 1-deoxynojirimycin is used as a calorie reducing agent, the effective amount as 1-deoxynojirimycin is at least 0.0001% by weight, preferably 0.001-0.5% by weight, to the carbohydrate contained in food.

The carbohydrate used in the present invention is usually contained in food and there is no limitation at all therefor but, preferably, a di- or higher polysaccharide is used.

Now an action mechanism of 1-deoxynojirimycin or a preparation derived from plants of family Moraceae containing 1-deoxynojirimycin for reducing the calorie of food will be explained.

Carbohydrate in food which we take such as starch moves to stomach together with being hydrolyzed by the action of ptyalin (α-amylase) in saliva. In the stomach, digestion proceeds in the part where it is not miscible with gastric juice whereupon about 70% thereof are hydrolyzed. When it is discharged from stomach to duodenum, it is decomposed by α-amylase in pancreatic juice to oligosaccharides such as α-limited dextrin and maltotriose and further to disaccharides such as sucrose and maltose. The hydrolyzed disaccharides are transferred to small intestine and hydrolyzed to monosaccharides such as glucose and fructose as a result of hydrolysis with an enzyme which decomposes disaccharides (α-glucosidase) existing in fine ciliary membrane surface (brush border) at the upper area of small intestine. They are absorbed with the body through the intestinal walls. The absorbed monosaccharides are changed to acetyl-CoA and then oxidized to water and carbon dioxide by a citric acid cycle and an oxidative phosphorylation. During that process, adenosine triphosphate is produced and utilized as an energy source for exercise of muscles, maintenance of body temperature, etc. It is said that, when the absorption rate into body is taken into consideration, the energy converted from 1 g of carbohydrate contained in food is 4 kcal. In a calorie calculation of food, that value is used.

However, when we orally take the food together with 1-deoxynojirimycin contained in the plants of family Moraceae, the carbohydrate in the food is digested by the above-mentioned process until arriving small intestine and is decomposed into disaccharides while, when 1-deoxynojirimycin is transferred to small intestine, it is bonded to α-glucosidase which is an enzyme existing in the brush border of epithelial cells of small intestine. 1-Deoxynojirimycin has far higher binding affinity than disaccharides such as sucrose and maltose have and it inhibits the bonding of those disaccharides with an enzyme in a competitively antagonistic manner. Thus, α-glucosidase is no longer able to hydrolyze the disaccharides. As a result, large amounts of disaccharides are not digested in and absorbed with small intestine but is carried to large intestine and excreted. Therefore, adenosine triphosphate which is to be produced from carbohydrate contained in the food is not produced and its conversion to energy does not take place as well. That means a reduction of calorie of the food. That is an action mechanism of contribution of 1-deoxynojirimycin and 1-deoxynojirimycin-containing food/drink derived from plants of family Moraceae to reduction of calorie of the food.

In order to effectively express such an action, it is desirable that 1-deoxynojirimycin or a 1-deoxynojirimycin-containing mulberry tea, powder or extract is firstly ingested orally and, after that, carbohydrate or food containing it is ingested. However, there is no limitation thereto but the action can be effectively achieved even by ingestion of food/drink in which carbohydrate and 1-deoxynojirimycin or a 1-deoxynojirimycin-containing mulberry tea, powder or extract are contained unitedly.

For example, in order to express such an action by drinking the mulberry tea during the meal, ingestion of it immediately before or during the meal is effective.

A calorie reducing agent derived from 1-deoxynojirimycin or from 1-deoxynojirimycin-containing plants of family Moraceae may be conveniently used by making into a form of powder or granules prepared by means of spray-drying together with an excipient such as a water-soluble high-molecular compound, e.g. dextrin, CMC, gumarabic and gelatin, or may be used by making into a form of tablets or capsules as well.

In the calorie reducing food/drink according to the present invention, there is no particular limitation for the food group. For example, the food/drink can be prepared by adding 1-deoxynojirimycin or a substance derived from 1-deoxynojirimycin-containing plant of family Moraceae to wheat flour products represented by bread, macaroni, spaghetti and noodles; instant food represented by instant noodles, instant noodles in a cup, pre-cooked food in a retort pouch, instant soup and stew and instant miso soup/clear soup; processed agricultural products represented by jam, marmalade, boiled beans and cereals (processed grain products); processed marine products represented by tsukudani (food boiled down in soy sauce), fish meat ham and sausage and fish meat paste products; processed animal meats represented by animal ham/sausage and paste; milk products represented by milk beverage, yogurt, lactic acid bacillus drink, cheese, dry milk, ice cream and cream; basic and compounded condiments represented by soy sauce, miso (fermented soy paste), sauce, processed tomato condiment, mirin (sugar-added rice wine), vinegar, instant curry seasoning, gravy, dressing, sauce for noodles and spice; confectionery represented by caramel/candy, chewing gum, chocolate, cookie/biscuit, cake/pie, snack cracker, Japanese cake, rice cake and bean cake and dessert cake; refreshing beverages represented by carbonated beverage, natural fruit juice, fruit juice beverage, refreshing beverage containing fruit juice, sarcocarp beverage, fruit beverage containing fruit grains, vegetable beverage, soybean milk and beverage containing the same, coffee drink, tea drink, powdery drink, concentrated drink, sports drink, nutritive health drink and alcoholic beverage; frozen dessert represented by ice-lolly and sherbet; sweeteners represented by honey, maple syrup and sugar; baby food; furikake (fish flour); laver preparation for ochazuke (boiled rice served with hot tea); and various health foods such as a beverage containing lactic acid bacilli or Bifidus bacilli and yogurt containing Bifidus bacilli.

The calorie reducing feed for animals according to the present invention can be prepared by adding 1-deoxynojirimycin or a substance derived from plants of family Moraceae containing 1-deoxynojirimycin not only to commonly used pet food but also to food groups mentioned in the above calorie reduced food/drink.

### EXAMPLES

The present invention will now be specifically illustrated by way of the following Examples although the present invention is not limited thereto.

### Preparation Example 1

Distilled water (20 liters) was added to 10 kg of dried powder of mulberry leaves (Ichinose) and an extraction was carried out with warming and stirring.

To this extract were added 20 liters of ethanol, the mixture was allowed to stand for one night and, after removing the resulting precipitate by means of centrifugation, it was concentrated in vacuo. The concentrate was freeze-dried to give 1.9 kg of a mulberry leaf extract. This freeze-dried substance was dissolved in 10 liters of distilled water and passed through a column of Dowex 50×4 (H⁺ type) and the column was well washed with distilled water to remove acidic and neutral parts and then eluted with 1 N NH₄OH solution to give a basic part. This basic solution was concentrated in vacuo to give 175 g of a 1-deoxynojirimycin-containing mulberry leaf extract.

It was confirmed that this extract contained 13 g of 1-deoxynojirimycin by means of a high pressure liquid chromatography.

After that, 75 g of this extract was passed through a column of Amberlite CG-50 (NH₄⁺ type) and eluted with distilled water to collect a fraction containing 1-deoxynojirimycin together with a confirming step by means of a thin layer chromatography. After the eluate was concentrated in vacuo and dried, it was dissolved in methanol and allowed to stand whereupon crystals (5.2 g) were separated out therefrom. They were purified by means of recrystallization whereupon melting point (194-195° C.), elementary composition (C₆H₁₃NO₄), ¹³C-NMR spectrum and mass analysis data of the resulting crystals were found to be identical with those of the authentic sample of 1-deoxynojirimycin.

### Example 1

1-Deoxynojirimycin and a mulberry leaf extract prepared in Preparation Example 1 were used to measure the amount necessary for 50%-inhibiting the activity of α-glucosidase (sucrase) existing in small intestine of rat (IC₅₀). As to the α-glucosidase, a sucrase solution which was partially purified from small intestinal mucous membrane (brush edge membrane) of rat was used. Namely, the mucous membrane of small intestine was exfoliated using a slide glass, homogenized at 4° C. for 5 minutes with a phosphate buffer and subjected to an ultrasonic treatment for 5 minutes. The resulting solution of smashed mucous membrane was centrifuged and the supernatant liquid thereof was used as an enzyme solution (sucrase). A 5 mM sucrose solution was used as a substrate, an enzyme solution and a predetermined amount of a 1-deoxynojirimycin solution or a mulberry extract were added and the reaction was carried out at 37° C. for 60 minutes and then stopped by a treatment at 100° C. for 5 minutes. Amount of glucose in the reaction solution was measured by a biochemical automatic analyzer.

As shown in Table 1, the result revealed that 1-deoxynojirimycin and a mulberry extract showed a very strong inhibiting action to sucrase.

TABLE 1

| | IC₅₀ to Sucrase | |
|---|---|---|
| 1-Deoxynojirimycin | 0.05 | μg/ml |
| Mulberry Leaf Extract | 7 | μg/ml |

### Preparation Example 2

Mulberry leaves (Ichinose) was cut in a width of about 1 cm and dried mulberry leaves were prepared using a tea manufacturing machine of a Date type. Conditions for the preparation were as follows. Thus, the mulberry leaves were steamed using a steamer, treated in a rough rubbing machine and a medium rubbing machine at 75° C. for 40 minutes and at 60-70° C. for 30 minutes, respectively and dried at 60-70° C. for 25 minutes using an air-permeable drier. The dried mulberry leaves manufactured as such were roasted using a roaster to prepare a mulberry tea.

### Example 2

Healthy three males taking no pharmaceuticals were asked to stop taking meals after 10 p.m. and, in the morning of the next day, mulberry leaf powder or a mulberry tea was ingested to them together with sugar (50 g) . After 30 minutes, blood was collected from the tip of a finger and the blood glucose level therein was measured using a plain blood sugar measuring apparatus (Glutest Ace manufactured by Sanwa Kagaku Kenkyusho) The result was found to be that, as shown in Table 2, in the case of ingestion of mulberry leaf powder or mulberry tea as compared with the control, an effect of suppressing the blood glucose level was noted and the absorbed calorie was little as well.

TABLE 2

| | Blood Glucose | Calorie |
|---|---|---|
| Samples Used | Level (mg/dl) | (kcal) |
| Control (50 g sugar only) | 213 ± 15 | 200 |
| Mulberry Leaf Powder | 163 ± 13 | 154 |
| (0.8 g ingested before 15 minutes) | | |
| Mulberry Leaf Powder | 129 ± 15 | 122 |
| (1.6 g ingested before 15 minutes) | | |
| Mulberry Leaf Powder | 135 ± 10 | 126 |
| (0.8 g ingested at the same time) | | |
| Mulberry Tea | 153 ± 18 | 142 |
| (3 g tea leaf ingested before 15 minutes) | | |

### Example 3

To 540 g of granular sugar and 440 g of starch syrup were added 5 g, 10 g, 20 g and 40 g each of the mulberry extract of Preparation Example 1 to prepare candies. Healthy 3 males taking no pharmaceuticals were asked to stop taking meals after 10 p.m. and, in the morning of the next day, 10 g of the candy were ingested to them. After 30 minutes, blood was collected from the tip of a finger and the blood glucose level therein was measured using a plain blood sugar measuring apparatus (Glutest Ace manufactured by Sanwa Kagaku Kenkyusho). The result was found to be that, as shown in Table 3, in the case of ingestion of the candy added mulberry leaf extract as compared with the case where no mulberry leaf extract was added to candy (control sample), an effect of suppressing the increase of blood glucose level was noted in a corresponding manner to an increase of the added amount of the mulberry leaf extract and the absorbed calorie was little as well.

TABLE 3

| | Blood Glucose | Calorie |
|---|---|---|
| Samples | Level (mg/dl) | (kcal) |
| No Mulberry Leaf Extract Added (control) | 150 ± 15 | 40 |
| 5 g Mulberry Leaf Extract | 143 ± 13 | 38 |
| 10 g Mulberry Leaf Extract | 128 ± 11 | 34 |
| 20 g Mulberry Leaf Extract | 110 ± 12 | 29 |
| 40 g Mulberry Leaf Extract | 105 ± 9 | 28 |

### Example 4

Biscuit, chewing gum, chocolate, candy and mulberry drink compounded with the powdered mulberry leaves or the mulberry leaf extract were manufactured according to a conventional method. In all of the food/drink manufactured as such, no abnormal taste or the like was noted.

| [Biscuit] | | |
|---|---|---|
| Sugar | 175 | parts by weight |
| Salt-free butter | 150 | parts by weight |
| Total egg | 150 | parts by weight |
| Wheat flour | 500 | parts by weight |
| Perfume | q.s. | parts by weight |
| Powdered mulberry leaves | 25 | parts by weight |
| Total | 1000 | parts by weight |
| [Chewing Gum] | | |
| Gum base | 200 | parts by weight |
| Sugar | 550 | parts by weight |
| Glucose | 80 | parts by weight |
| Starch syrup | 145 | parts by weight |
| Glycerol | 5 | parts by weight |
| Perfume | q.s. | parts by weight |
| Mulberry leaf extract | 20 | parts by weight |
| Total | 1000 | parts by weight |
| [Chocolate] | | |
| Cacao mass | 440 | parts by weight |
| Sugar | 430 | parts by weight |
| Cacao butter | 110 | parts by weight |
| Lecithin | q.s. | parts by weight |
| Perfume | q.s. | parts by weight |
| Mulberry leaf extract | 20 | parts by weight |
| Total | 1000 | parts by weight |
| [Candy] | | |
| Granular sugar | 540 | parts by weight |
| Starch syrup | 440 | parts by weight |
| Water | a little | parts by weight |
| Sour taste agent | q.s. | parts by weight |
| Perfume | q.s. | parts by weight |
| Coloring agent | q.s. | parts by weight |
| Mulberry leaf extract | 20 | parts by weight |
| Total | 1000 | parts by weight |
| [Mulberry Drink] | | |
| Invert sugar | 77 | part(s) by weight |
| Sugar | 65 | part(s) by weight |
| Mulberry fruit juice | 40 | part(s) by weight |
| Sour taste agent | 1 | part(s) by weight |
| Perfume | 1 | part(s) by weight |
| Vitamin C | 0.5 | part(s) by weight |
| Mulberry leaf extract | 5 | part(s) by weight |
| Water was added to make | 1000 | parts by weight |
| [Yogurt Compounded with Bifidus Bacilli] | | |
| Yogurt (Bulgarian type) | 450 | parts by weight |
| Water | 200 | parts by weight |
| Stabilizer | 4 | parts by weight |
| Powdered sugar | 45 | parts by weight |
| Mulberry fruit juice | 286 | parts by weight |
| Perfume | q.s. | parts by weight |
| Bifidus bacilli powder | q.s. | parts by weight |
| Mulberry leaf extract | 15 | parts by weight |
| Total | 1000 | parts by weight |

### Example 5

Dry dog food compounded with a mulberry leaf extract was manufactured by a conventional method. When this was given to shibainu (a Japanese dog of medium size), there was no difference at all in terms of snapping as compared with a commercially available pet food.

| | | |
|---|---|---|
| Wheat flour | 150 | parts by weight |
| Corn | 150 | parts by weight |
| Skim soybean | 150 | parts by weight |
| Steamed blood-containing bonito meat | 220 | parts by weight |
| Steamed blood-containing tuna meat | 200 | parts by weight |
| Chicken | 100 | parts by weight |
| Vitamin/mineral mixture | 10 | parts by weight |
| Calcium phosphate | 10 | parts by weight |
| Mulberry leaf extract | 10 | parts by weight |
| Total | 1000 | parts by weight |

What is claimed is:

1. A method of reducing a calorie of food or beverage, which comprises administering an effective amount of 1-deoxynojirimycin or an analogue thereof as the effective ingredient to a person or animal.

2. The method as claimed in claim 1, in which an effective amount of 1-deoxynojirimycin or an analogue thereof is incorporated into food or beverage.

3. The method as claimed in claim 1, in which a mulberry tea manufactured from a plant of family Moraceae containing 1-deoxynojirimycin or an analogue thereof is administered.

4. The method as claimed in claim 1, in which a powdered plant of family Moraceae containing 1-deoxynojirimycin or an analogue thereof is administered.

5. The method as claimed in claim 1, in which an extract of a plant of family Moraceae containing 1-deoxynojirimycin or an analogue thereof is administered.

6. The method as claimed in claim 1, in which a carbohydrate is further administered.

7. The method as claimed in claim 1 or 6, in which the administration is effected together with food or beverage.

8. A calorie reducing composition comprising a carbohydrate and at least one substance selected from the group consisting of 1-deoxynojirimycin, an analogue thereof, a mulberry tea manufactured from a plant of family Morac containing 1-deoxynojirimycin or an analogue thereof, a powdered plant of family Moraceae containing 1-deoxynojirimycin or an analogue thereof and an extract of a plant of family Moraceae containing 1-deoxynojirimycin or an analogue thereof.

9. A calorie reduced food or beverage comprising food or beverage and at least one substance selected from the group consisting of 1-deoxynojirimycin, an analogue thereof, a mulberry tea manufactured from a plant of family Morac containing 1-deoxynojirimycin or an analogue thereof, a powdered plant of family Moraceae containing 1-deoxynojirimycin or an analogue thereof and an extract of a plant of family Moraceae containing 1-deoxynojirimycin or an analogue thereof.

10. The food or beverage as claimed in claim 1, which further comprises a carbohydrate.
